(19) United States
(12) Patent Application Publication
(10) Pub. No.: US 2006/0246054 A1
(43) Pub. Date: Nov. 2, 2006

(54) Bromelain preparation and pharmaceutical composition containing the same

(75) Inventors: Yuh-Shuen Chen, Taichung (TW); Chien-Wei Hou, Taichung (TW); Wen-Hung Lin, Miao-Li Hsien (TW)

Correspondence Address: CHARLES E. BAXLEY, ESQ., 90 JOHN STREET, THIRD FLOOR, NEW YORK, NY, 10038, US

(73) Assignee: Come True Biotechnology Corp.

(21) Appl. No.: 11/119,861

(22) Filed: May 2, 2005

(51) Int. Cl. A61K 38/46

(52) U.S. Cl. 424/94.65

(57) ABSTRACT

The present invention relates to improved preparation of Bromelain in which the Bromelain is coated with an organic network polymer constructed by cross-linkage between organic acids and polysaccharides. The present invention also relates to a pharmaceutical composition comprising the present Bromelain preparation for treating inflammation, alleviating pains, and/or enhancing immuno-defense in a subject who needs such treatment.

### BACKGROUND OF THE INVENTION

1. Field of the Invention

The present invention relates to an improved preparation of Bromelain in which the Bromelain is coated with an organic network polymer constructed by cross-linkage between organic acids and polysaccharides. Particularly, the Bromelain is embedded in the organic network polymer through a crosslinking reaction primed by electron-beam irradiation. The present invention also relates to a pharmaceutical composition comprising the present Bromelain preparation for treating inflammation, alleviating pains, and/or enhancing immuno-defense in a subject who needs such treatments.

2. Related Prior Arts

Bromelain is a group of proteases extracted from the stem of pineapple. The fist therapeutic effect of Bromelain was found in anti-inflammation, see, for example, Seligman B., Angiology 13:508-510 (1962); and Kelly G S, Alt Med Rev 1(4):243-257 (1996). The other pharmalogical activities of Bromelain have been reported in literatures, such as reduction of thrombogenesis, anti-hypertension, regulation of immunofunctions, anti-microbial infections and inhibition of cancer cell growth, see, for example, Livio M et al, Drugs Exp Clin Res 4:49-53 (1978); Hale L, J Immuno 149:3809-3816 (1992); Chandler D s et al, Gut 43:196-202 (1998); and Taussig S J et al, Planta Medica 6:538-539 (1985).

For the feature of protein, Bromelain may be degraded or denatured in digestive tract such as stomach. In order to enhance the bioavailability, Bromelain is coated with acid-resisting coating or encapsulated in capsule for oral administration. Thus, the present invention provides a novel dosage form of Bromelain with improved pharmalogical effects, which is made by an easy prosess. In such Bromelain preparation, Bromelain is embedded in an organic network polymer, which will protect Bromelain from degradation in acidic environments.

### SUMMARY OF THE INVENTION

In one aspect, the present invention relates to a improved Bromelain preparation composed of Bromelain embedded in an organic network polymer constructed through a crosslinking reaction between organic acids and polysaccharides by electron-beam irradiation.

In a further aspect, the present invention relates to a process for manufacturing the improved Bromelain preparation, which comprising first cross-linking reaction between an organic acid and a polysaccharide by electron-beam irradiation to form an organic network polymer and second cross-linking reaction between the formed organic network polymer and Bromelain by electron-beam irradiation to embed the Bromelain into the organic network polymer.

In another aspect, the present invention relates to a pharmaceutical composition comprising the improved Bromelain preparation, which is used in anti-inflammation.

In still another aspect, the present invention relates to a pharmaceutical composition comprising the improved Bromelain preparation, which is used in alleviating pains.

In still another aspect, the present invention relates to a pharmaceutical composition comprising the improved Bromelain preparation, which is used in enhancing immuno-defense.

### BRIEF DESCRIPTION OF THE DRAWINGS

FIG. 1 shows the Interleukin-β (IL-1β) concentration in serums from differently treated groups (N=8) rats for each group) after bacterial LPS stress (dose 2.5 mg/kg, 24 h). The control represents the animals unchallenged with bacterial LPS. The MP represents animals treated with a marketing product.

FIG. 2 shows the Interleukin-6 (IL-6) concentration in serums from differently treated groups (N=8) rats for each group) after bacterial LPS stress (dose 2.5 mg/kg, 24 h). The control represents the animals unchallenged with bacterial LPS. The MP represents animals treated with a marketing product.

FIG. 3 shows the Tumor necrosis factor-α (TNF-α) concentration in serums from differently treated groups (N=8) rats for each group) after bacterial LPS stress (dose 2.5 mg/kg, 24 h). The control represents the animals unchallenged with bacterial LPS. The MP represents animals treated with a marketing product.

FIG. 4 shows the cytotoxicity of treatments (including LPS (dose 1.0 μg/ml, 24 h), Bromelain preparation 10 μg/ml+LPS, Bromelain preparation 50 μg/ml+LPS, Bromelain preparation 100 μg/ml+LPS) to microglia BV-2 cells. The control represents the cells un stimulated with bacterial LPS.

FIG. 5 shows the amount of prostaglandin PGE₂ released by LPS-stimulated (dose 1.0 μg/ml, 24 h) microglia BV-2 cells with Bromelain preparation (0 μg/ml, 10 μg/ml, 50 μg/ml, or 100 μg/ml). The control represents the cells unstimulated with bacterial LPS.

FIG. 6 shows the COX-2 expression in LPS-stimulated (dose 1.0 μg/ml, 4 h) microglia BV-2 cells with Bromelain preparation (0 μg/ml, 10 μg/ml, 50 μg/ml, or 100 μg/ml). The control represents the cells unstimulated with bacterial LPS.

FIG. 7 shows the comparative PGE₂ release tests of the Bromelain preparation and seven marketing Bromelain products (A to G) at same dosage (50 μg /ml).

### DETAILED DESCRIPTION OF THE PREFERRED EMBODIMENTS

Inflammation is an important signal for human body, which indicates that something is wrong. When body tissues are damaged regardless it is due to biological, physical or chemical factors, macrophages around the damaged tissues will be activated to eliminate the foreign objects. At the same time, they will also release some factors to activate other immuno-defense systems, such as, nitric oxide, tumor necrosis factor, interleukin, granulocyte-monocyte colony-stimulating factor, granulocyte colony-stimulating factor, and monocyte colony-stimulating factor. The concentration of the above factors was elevated in inflamed tissue.

In 1975 Carswell and colleague reported that by injecting mice with bacterial LPS, a tumor-killing factor could be detected in the serum, and they called it tumor necrosis factor (TNF). Later Shalaby (1985) named the TNF produced by macrophage TNF-α, and the lymphotoxin that produced by T lymphocyte as TNF-β. TNF-α is produced by monocyte and macrophage, and bacterial LPS served as a strong stimulant. Bacterial endotoxin frequently caused severe ailment in digestive system, and bacterial LPS is a major endotoxin. Animal studies indicate that LPS will delay Gastric Emptying and it is related to the induction of the immune responses. When body was stimulated by LPS, it would induce the formation of TNF-α, IL-1β, IL-6, and other factors to participate defense and repair process. However, too much these factors will also cause undesirable effect on our body, for example, too much TNF-α will cause organ prostration, toxic shock or even death. It has been shown that the addition of TNF-α antibody can effectively prevent the onset of detrimental shock syndrome caused by endotoxin. Researches indicated that the formation of TNF-α, IL-1β, and IL-6 were related. LPS will induce the synthesis of TNF-α which is then induced the formation of IL-1β, and then the later will induce the formation of IL-6.

Metabolism of arachidonic acid by the type-2 cyclooxygenase (COX-2) pathway produces prostaglandins and thromboxanes. Different kinds of prostaglandin are produced by different type of cell. For example, monocytes and marcophages produce large quantities of PGE2 and PGF2; neutrophils produce moderate amounts of PGE2; mast cells produce PGD2. Prostaglandins have diverse physiological effects, including increased vascular permeability, increased vascular dilation, and induction of neutrophil chemotaxis associated with inflammation responses.

From the inhibitive activities on the release of TNF-α, IL-1β, IL-6 and prostaglandin and the expression of COX-2 as described in following examples, it is demonstrated that the present Bromelain preparation exhibits a significantly improved effct in anti-inflammation.

### EXAMPLES

The other aspects and features of the invention will become apparent in the descriptions of following examples. These examples are given for illustration of the invention and are not intended to be limiting thereof.

### Example 1

### Preparation of the Improved Bromelain Preparation

Starch and oragnic acid were mixed at 2:1 by weight, the mixture was heated at 80° C. for 5 min with stirring. The stirred mixture was irradiated with electron beam of 15 kgy for 3 seconds to form an organic network polymer. The mixture of organic network polymer was neutralized with appropriate amount of NaHCO₃ to about pH 7.

The neutralized mixture was added same weight of Bromelain at 30° C. then stirred for 3 min. The mixture was subjected the second irradiation with electron beam of 15 kgy for 3 seconds to embed Bromelain into the organic network polymer and obtained the Bromelain preparation.

The Bromelain preparation was tested for residual activity in acidic envirronment of pH 3, 4, 5, 6, and 7 at 30° C. for 2 hours. The remaining activities at pH 3, 4, 5, 6 and 7 were 82%, 86%, 92%, 95%, and 96%, compared to the residual activities of uncoated Bromelain of less than 50% after treated at pH 3 and 4 at 30° C. for 2 hours.

### Example 2

### Anti-Inflammation Effects of the Improved Bromelain Preparation

A. Animal Tests

Male SD rats, weight about 250 g each, were purchased from BioLASCO Taiwan Co., Ltd. Rats were kept under 23° C., with 12 hours day/night cycle, and fed with regular diet. Drinking water was pre-treated with reverse osmotic technique. Five sets of rats were treated differently, and each set contain 10 rats. In the controls set, rats were held under regular diet through the experiment. In lipopolysaccharide (LPS) treated set, rats were held under regular diet and then Escherichia coli LPS (2.5 mg/kg) was injected into the abdominal cavity of each rat. In the remaining 3 sets of rat, before the injection of LPS, their diets were supplemented with different rates of the improved Bromelain preparation (10 mg/kg, 50 mg/kg or 100 mg/kg) for 7 days. After LPS injection, rats were kept on a 24h-fast. Blood were then drawn from celiac vein of each rat before and after stress treatment for immunological study. The data were analyzed using one-way analysis of variance (ANOVA).

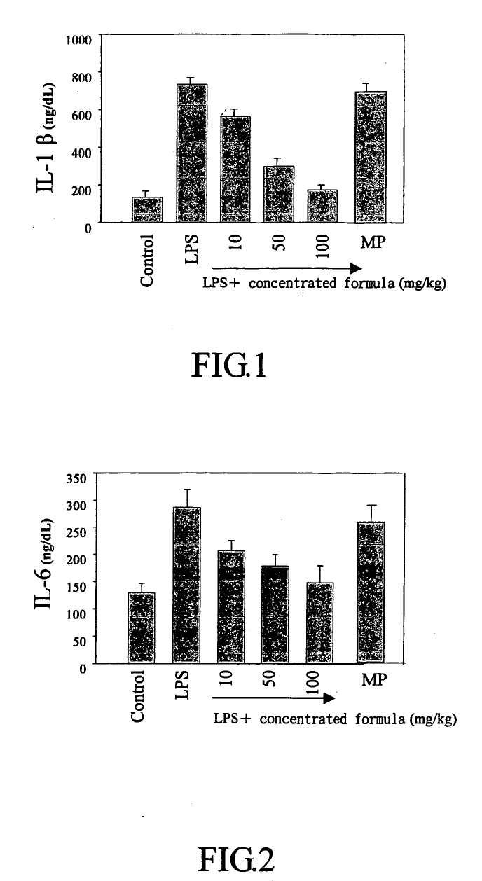

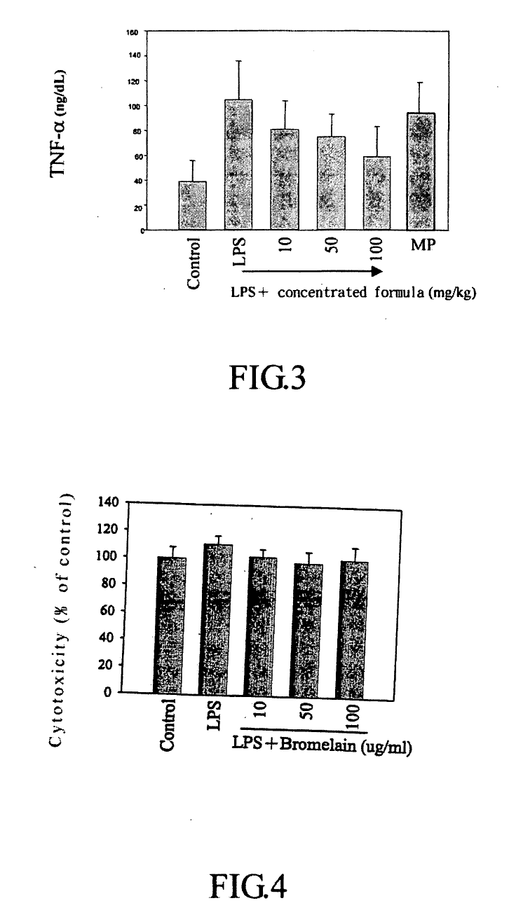

The concentrations of Interleukin-1 (IL-1), Interleukin-6 (IL-6) and Tumor necrosis factor-α (TNF-α) were assayed using ELISA. As shown in FIG. 1 to 3, feeding of the improved Bromelain preparation could effectively reduced the serum contain of TNF-α, IL-1β, and IL-6 when the rats were challenged with bacterial LPS. The reduction of serum TNF-α, IL-1β, and IL-6 concentration by the improved Bromelain preparation is dose-dependent. In the serum immunoassay of IL-1β (FIG. 1), feeding of low dose (10 mg/kg) of the improved Bromelain preparation reduced IL-1β content in serum from LPS-challenged rats by 31%, comparing to the LPS group. Similarly at low dose, the concentration of serum IL-6 and TNF-α were reduced by 41% and 40% respectively, comparing to the LPS group (see FIGS. 2 and 3). Feeding of medium and high dose (50 and 100 mg/kg) of the improved Bromelain preparation exhibited significant anti-inflammation effects in treated animals, as shown in the following table 1.

TABLE 1
Changes of Interleukin-1β concentration
in serums from differently treated rats.

| | | IL-1 β | IL-1 β |
|---|---|---|---|
| | | (interlukin-1) | (interlukin-1) |
| | | Original | After stress |
| Groups | | (ng/dL) | (ng/dL) |
| 1. | Control | 119 ± 12 | 136 ± 23 |
| 2. | LPS (24 h) | 111 ± 20 | 735 ± 35 |
| 3. | LPS (24 h) + Bromelain | 116 ± 14 | 565 ± 38 |
| | preparation 10 mg/kg | | |
| 4. | LPS (24 h) + Bromelain | 104 ± 17 | 301 ± 43 |
| | preparation 50 mg/kg | | |
| 5. | LPS (24 h) + Bromelain | 125 ± 11 | 174 ± 26 |
| | preparation 100 mg/kg | | |
| 6. | Marketing product 100 mg/kg | 112 ± 16 | 712 ± 32 |

LPS: E. coli-lipopolysaccharides (dose, 2.5 mg/kg)
Each group N = 10

Accordingly, these results suggest an effect in anti-inflammation of the improved Bromelain preparation.

We used microglia BV-2 cells as model to investigate the cytotoxicity and inhibitive effects in prostaglandin PGE₂ release and COX-2 expression of the improved Bromelain preparation. BV-2 cell line was maintained in DEME supplemented with 10% FBS and antibiotics at 37° C. under 5% CO₂. Confluant cultures were passed by trypsinization. For experiments, cells were washed twice with warm DMEM (without phenol red), then treated in serum-free medium. In all experiments, cells were treated with the Bromelain preparation in 1×PBS (phosphate-buffered saline).

Cytotoxicity was determined by measuring the release of lactate dehydrogenase (LDH). BV-2 cells were preincubated in 24-well plates at a density of 5×10⁵ cells per well for 24 hours, then washed with phosphate-buffered saline (PBS). BV-2 cells with various concentration of the Bromelain preparation were treated with LPS for 24 hours and the supernatant was used to assay LDH activity. The reaction was initiated by mixing 0.1 ml of cell free supernatant with potassium phosphate buffer containing NADH and sodium pyruvate in a final volume of 0.2 ml to 96-well plate. The rate of absorbance value was read at 490/630 nm on an automated SpectraMAX 340 microtiter plate reader. Data were expressed as the mean percent viable cell vs. LPS control.

The result was shown in FIG. 4. From the result of LDH release assay, it was demonstrated that the present Bromelain preparation has no significant cytotoxicity to BV-2 cells, and meams that it is harmless to BV-2 cells.

Prostaglandin PGE₂ release by LPS-stimulated BV-2 cell with various concentrations of the Bromelain preparation plus 1 μg/ml LPS after 24 hours treatment was measured by ELISA immunoassay kit (R&D system, Minneapolis, USA). The linear range of the assay was from 10 to 1000 μg/ml. BV-2 cell suspensions were diluted or concentrated to achieve values that fall within the linear ranges of the assays. The PGE₂ values were read at 450/570 nm on an automated SpectraMAX 340 microtiter plate reader. Data were expressed as the mean percent viable cell compared to the control.

As shown in FIG. 5, treatment with 10 μg/ml of the Bromelain preparation reduced the release of PGE₂ by LPS-stimulated BV-2 cells by 27%, compared to the LPS control group. Treatments with 50 μg/ml and 100 μg/ml of the Bromelain preparation significantly reduced the release of PGE₂ by LPS-stimulated BV-2 cells by 50% and 80% respectively, compared to the LPS control group.

For detecting the effects of the Bromelain preparation on cyclooxgenase-2 (COX-2) activity and expression in LPS-stimulated BV-2 cells, total RNA was purified from BV-2 cell with various concentrations of the Bromelain preparation plus 1 μg/ml LPS after 4 hours incubation and using TRIzol (GIBCO BRL) following the protocol recommended by the manufacturer. COX-2 expression (real-time RT-PCR) was analysed by real time quantitative RT-PCR assay. Total RNA (0.5 μg) was reverse transcribed with random primers with M-MLV reverse transcriptase, in the presence of RNase Out™ (Invitroben, USA). One hundred nanograms reverse transcribed RNA was primed with specific oligonucleotides for COX-2:

COX-2:
(5′-GAACATTGTGAACATCCCC-3′
and
5′-GGTGGCATACATCATCAGACC-3′);
β-actin
(5′-GAACATTGTGAACATCCCC-3′
and
5′-GGTGGCATACATCATCAGACC-3′).

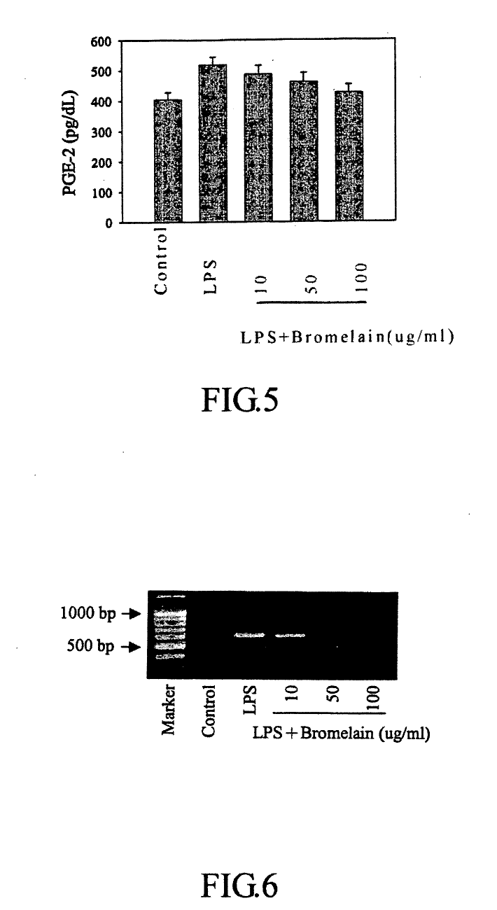

PCR was accomplished with ABI PRISM 7000 Deetection System (Applied Biosystems, USA). The PCR product was visualized by electrophoresis in 2% agarose gel, staining with ethidium bromide. Verification of specific genes was established by their predicted size under UV light. The result was shown in FIG. 6. It suggested that the Bromelain preparation supressed the expression of COX-2 and further inhibited the biosynthesis and release of prostaglandin and inflammation-related cytokines such as IL-1, IL-6, TNF-α and the like, acting as one kind of NSAID (nonsteroidal anti-inflammatory drug).

### Example 3

### Comparative Anti-Inflammation Tests of the Bromelain Preparation and Marketing Products

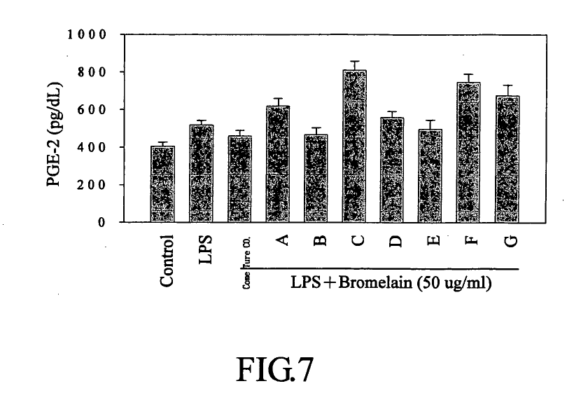

Following the prostaglandin PGE₂ release assay described in Example 2, the anti-inflammation effect of the Bromelain preparation was compared with seven marketing products at same dosage. As shown in FIG. 7, the Bromelain preparation of the invention (Cometrue) reduced the PGE₂ release by 50%. Comparing to the current marketing Bromelain products, only product B and product E exhibited the reducing effects in PGE₂ release by 44% and 18% respectively, and the other product showed no such inhibition effects in PGE₂ release by LPS-stimulated BV-2 cells. The present Bromelain preparation indeed exhibits improved anti-inflammation effects than the prior marketing Bromelain products.

1. An improved Bromelain preparation composed of Bromelain embedded in an organic network polymer constructed through a crosslinking reaction between an organic acid and a polysaccharide by electron-beam irradiation.

2. The improved Bromelain preparation of claim 1, in which the electron-beam irradiation is created by γ-ray.

3. The improved Bromelain preparation of claim 2, in which the electron-beam irradiation is created by ⁶⁰Co irradiation.

4. A process for manufacturing the improved Bromelain preparation of claim 1, which comprising first cross-linking reaction between an organic acid and a polysaccharide by electron-beam irradiation to form an organic network polymer and second first cross-linking reaction between the formed organic network polymer and Bromelain by electron-beam impact to embed the Bromelain into the organic network polymer.

5. The process of claim 4, in which the electron-beam impact is created by γ-ray of 5 kgy to 15 kgy.

6. The improved Bromelain preparation of claim 5, in which the electron-beam irradiation is created by ⁶⁰Co irradiation.

7. The process of claim 4, in which the organic acid is selected from lactic acid, malic acid and tartaric acid.

8. The process of claim 4, in which the polysaccharide is acidic starch.

9. The process of claim 4, in which the organic acid and polysaccharide are mixed at a ratio of about 1:2 by weight.

10. A pharmaceutical composition comprising the improved Bromelain preparation of claim 1, which is used in anti-inflammation.

11. A pharmaceutical composition comprising the improved Bromelain preparation of claim 1, which is used in alleviating pains.

12. A pharmaceutical composition comprising the improved Bromelain preparation of claim 1, which is used in enhancing immuno-defense.
